(19) United States
(12) Patent Application Publication
(10) Pub. No.: US 2002/0164678 A1
(43) Pub. Date: Nov. 7, 2002

(54) Method and device for laser cutting microscopic samples

(76) Inventors: Michael Ganser, Giessen (DE); Albrecht Weiss, Linden (DE); Ruediger Stenzel, Hilchenbach (DE)

Correspondence Address: FOLEY AND LARDNER, SUITE 500, 3000 K STREET NW, WASHINGTON, DC, 20007, US

(21) Appl. No.: 10/129,077

(22) PCT Filed: Apr. 10, 2001
(86) PCT No.: PCT/DE01/01414

(30) Foreign Application Priority Data
Apr. 13, 2000 (DE) 10018255.0

(51) Int. Cl. G01N 1/30; G01N 33/48; G06K 9/00; B23Q 15/00

(52) U.S. Cl. 435/40.5; 382/128; 83/72

(57) ABSTRACT

The invention relates to a method and a device for laser cutting microscopic samples. The device for laser cutting microscopic samples comprises a microscope (1) having at least one lens (6) for observing a sample (12) that is to be cut The lens (6) defines an optical axis (14) and a lens aperture (34). A laser (4) is also connected to the microscope (1). The laser (4) generates a laser beam (41) that is injected into the lens (6) by means of at least one optical system (16). A diaphragm (18) is provided, which generates a dimmed laser beam (4b), whereby the laser aperture (36) generated by the lens (6) is smaller than the lens aperture (34) of the lens (6) itself.

The invention relates to a method for laser cutting microscopic samples.

In addition, the invention relates to a device for laser cutting microscopic samples, the device comprising a microscope having at least one objective for observing a sample to be cut, the objective defining an optical axis and an objective aperture, a laser which produces a laser beam, and at least one optical system which injects the laser beam into the objective.

Diseases, such as cancer, have for a long time been identified by biopsies of tissue samples being performed in order to identify unnatural cells. The cells to be examined are isolated manually or mechanically by means of microdissection or by means of other complicated methods.

German Laid-Open Specification DE-196 16 216.5 describes such a method, the Laser Pressure Catapulting Method (LPC Method), as it is known. In this case, a part sample is cut out by means of a laser from a sample mounted on a transparent object slide. The removal of the cut-out part sample from the overall sample is carried out by means of an induced laser process in this method. For this purpose, a collecting device whose inner surface is coated with an adhesive is guided over the cut-out part sample by means of a carrier arm. This part sample is then subjected to a two-dimensional laser bombardment of suitable power, by means of which the cut-out part sample is catapulted upward out of the overall sample. The part sample detached in such a way is caught by the inner surface of the collecting device coated with adhesive and can then be fed to ongoing examinations. The laser pulse which is used to catapult the sample pieces can lead to damage to the tissue. In addition, sample particles detached from the cutting line on account of the cutting process can be deposited on the area of the sample to be examined. This problem arises primarily during the use of inverted microscopes.

In the case of the systems known from practise, the cutting quality of the laser may be adjusted by varying the laser intensity and the focal position. The aperture used for the laser light beam is determined by the objective aperture in the case of these known systems, it being necessary for said aperture in turn to be as large as possible for the maximum image quality. As already mentioned above, constant cutting quality is difficult to achieve in the case of the devices and methods of the prior art. The quality of the cuts depends firstly on the focal position of the preparation and its thickness and secondly on the laser intensity. The latter has to be varied by the users in order to optimize the cutting quality.

On the basis of this prior art, the invention is based on the object of configuring a device for laser cutting microscopic samples in such a way that an approximately constant cutting quality is ensured for a wide range of samples.

According to the invention, the achievement of this objective is characterized by the fact that a diaphragm is provided which produces a dimmed laser beam, a laser aperture produced by the objective being smaller than the aperture of the objective.

A further object of the invention is to describe a method for laser cutting microscopic preparations which permits an approximately constant cutting quality for a wide range of samples.

This object is achieved by a method which comprises the following steps:

a) introducing an object slide with a sample to be cut into a microscope which comprises at least one objective;

b) with the objective, determining an area of the sample to be cut out;

c) defining a cutting line around the area;

d) producing a dimmed laser beam by means of a diaphragm, so that the diameter of said beam is reduced in such a way that a laser aperture produced by the objective is smaller than the objective aperture of the objective itself; and

e) cutting the sample along the defined cutting line.

One advantage of the invention is that, as a result of the reduction in the laser aperture, the cone of laser light becomes slimmer, which leads to an increase in the depth of focus. Because of the greater depth of focus of the laser light, the requirement on the focusing accuracy is reduced and therefore leads to a uniform and narrow cutting channel.

Also advantageous in the configuration of the device according to the invention is that the magnitude of the objective aperture is maintained during the cutting operation. As a result, observing the sample with the full objective aperture is possible at any time. This ensures the best possible definition of the sample plane and the maximum image quality for assessing the sample. For the extremely detailed imaging and specific selection of areas of the sample, objective apertures up to about 0.8 are necessary. Of course, this necessitates a low depth of focus, so that it is possible to fix specifically on different planes in the sample. However, a low depth of focus is undesired for the operation of cutting with a laser beam. The invention now combines the relatively large objective aperture with a dimmed laser beam in such a way that the laser aperture produced by the objective is smaller than the aperture of the objective itself. The objective can be used for the simultaneous observation and cutting of the sample, with a constant opening.

According to a practical embodiment, the optical system contains a dichromatic splitter, which reflects the laser light and injects it into the objective and which, at the same time, lets the light of the observation beam path through to the eyepieces or to the camera.

In order to be able to control the laser cut, in particular with respect to the cutting quality, the invention further proposes that the laser cut be simultaneously controllable via an image-providing system, camera. If, during the evaluation of the images, it is established that either the preparation has not been severed completely during the laser bombardment or else the cutting geometry is inadequate, as a reaction of this simultaneous control of the laser cut, individual system parameters such as the laser intensity and/or the focal position of the laser beam and/or the size of the diaphragm in the laser beam can be adjusted via a computer. By means of this simultaneous control, the overall cutting time is shortened with improved quality.

Further features and advantages of the invention emerge from the following description of the associated drawing, in which an exemplary embodiment of a device according to the invention for laser cutting of microscopic samples is illustrated schematically by way of example. In the drawing:

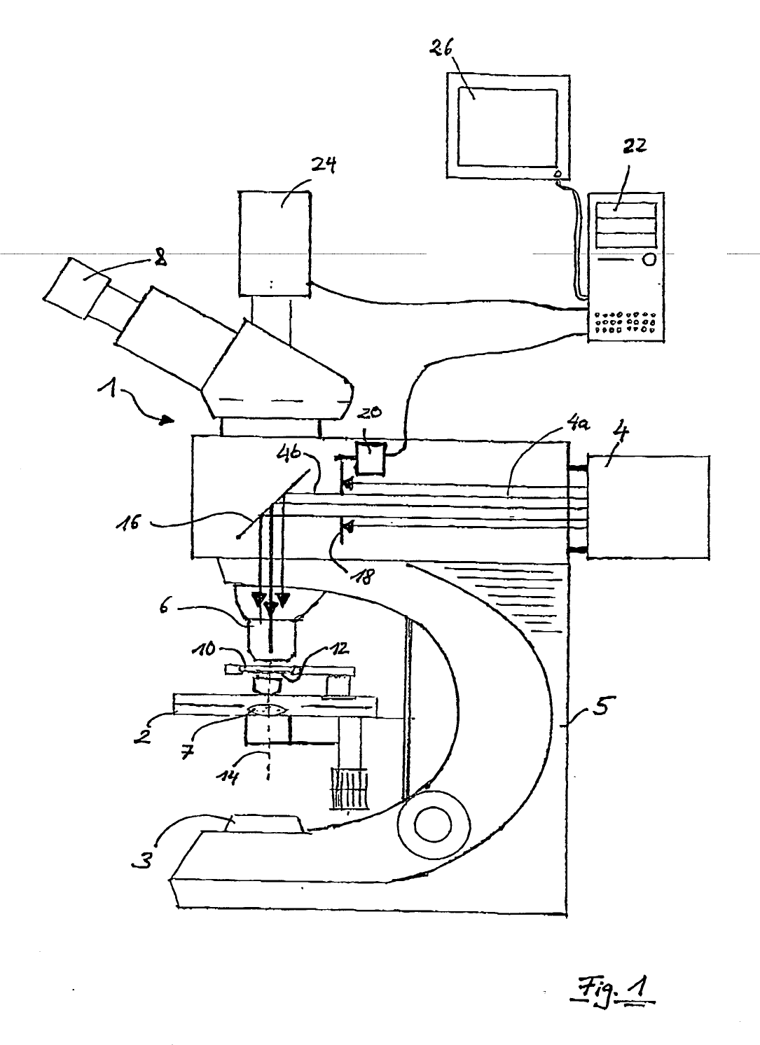

FIG. 1 shows a schematic side view of a device for laser cutting microscopic samples,

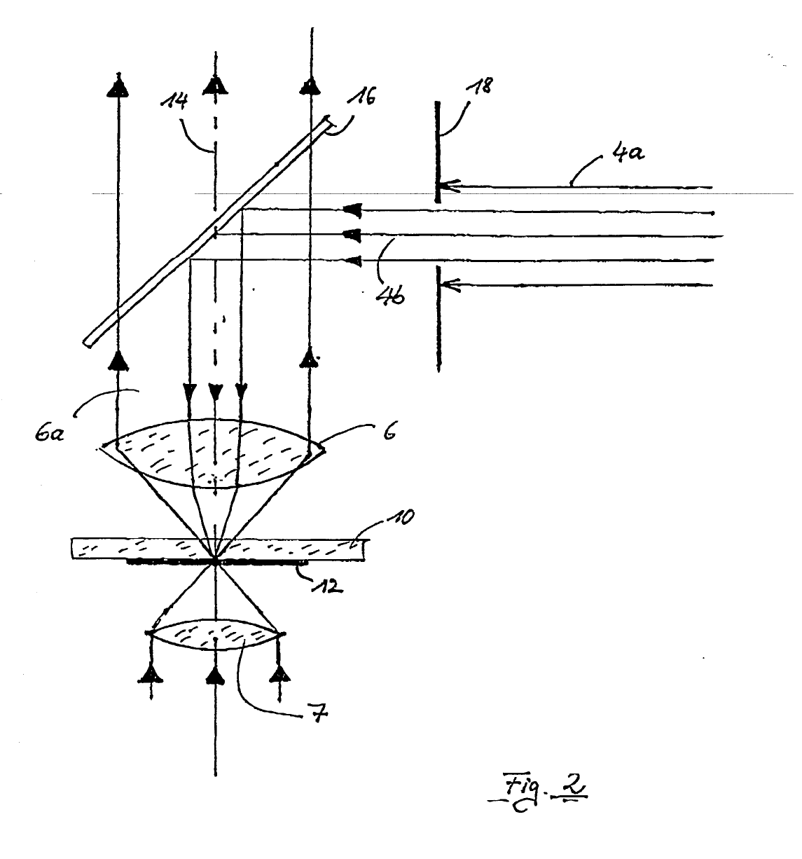

FIG. 2 shows the beam path in the area of the sample to be cut, and

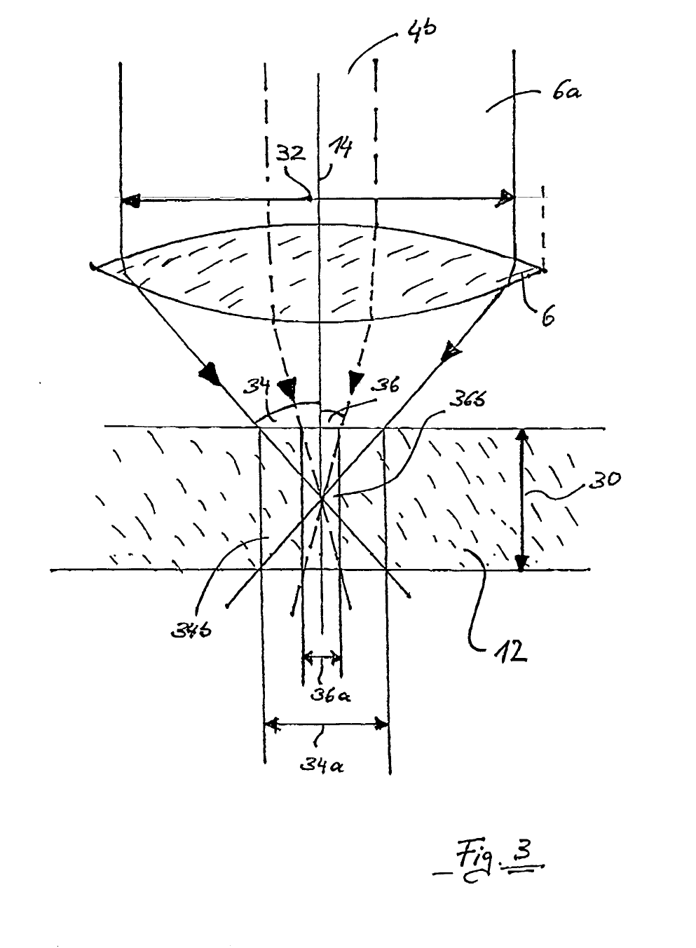

FIG. 3 shows a graphic representation of the cutting width as a function of the aperture of the laser beam.

The device illustrated in FIG. 1 for laser cutting microscopic samples a microscope 1, which is provided with a working table 2 to hold an object slide 10. A sample 12 to be examined and to be cut is fitted to the object slide 10. Also provided is an illumination system 3 and a laser 4, which produces a laser beam 4a which is used to cut the sample 12.

The microscope 1 illustrated is a microscope in which the illumination system 3 is arranged on the microscope stand 5 underneath the working table 2 and the sample 12. An objective 6 of the microscope 1 is arranged above the working table 2 and the sample 12. The objective 6 defines an optical axis 14, on which the illumination system 3 is likewise arranged. However, laser cutting can of course also be carried out with inverse microscopes, in which the illumination system 3 is then arranged above the working table 2 and the at least one objective 6 is arranged underneath the working table 2.

In the exemplary embodiment disclosed in FIG. 1, the light emitted by the illumination system 3 is directed from below, via a condenser lens 7, onto the object slide 10 and sample 12 arranged on the working table 2. The light penetrating the sample 12 passes to the objective 6 of the microscope 1. Within the microscope 1, the light is led via lenses and mirrors (not illustrated) to at least one eyepiece 8 of the microscope 1, through which an operator can observe the sample arranged on the working table 2.

In the stand 5 of the microscope 1, an optical system 16 is provided on the optical axis of the objective 6. The optical system 16 can be, for example, a dichromatic splitter. In addition, it is conceivable that the optical system 16 consists of a plurality of optical components. This is the case when the laser 4 has to be deflected around a plurality of corners. Also provided in the laser beam 4a is a diaphragm 18, with which the diameter of the laser beam can be restricted in an appropriate way. The diaphragm 18 can be designed, for example, as a fixed diaphragm. In this case, a plurality of fixed diaphragms are arranged in an appropriate way, for example on a turret disk, in order to move the required diaphragm 18 into the beam path. The method can be carried out manually by the user or by a motor. In the embodiment illustrated in FIG. 1, the diaphragm 18 is designed as a vario diaphragm, for example as an iris diaphragm, whose diameter is controlled via a motor 20. The motor 20 receives the necessary control signals for adjusting the required diaphragm diameter from a computer 22.

The microscope 1 is also provided with a camera 24, which records an image of the sample 12 to be cut. This image can be displayed on a monitor 26, which is connected to the computer 22. The system comprising computer 22, camera 24 and monitor 26 can be used for the purpose that the cutting operation by the laser 4 can be observed and monitored. In addition, on the monitor 26, by means of a mouse pointer, it is possible to move around the area of the sample 12 that is to be cut out. The cutting operation is then carried out by the laser 4 along the cutting line identified in this way.

FIG. 2 shows the beam path in the area of the sample 12 to be cut. The laser beam 4a coming from the laser 4 has its diameter restricted by the diaphragm 18. After the diaphragm 18, a dimmed laser beam 4b with a smaller diameter emerges. The laser beam 4b strikes the optical system 16, which is designed as a dichromatic splitter, and as a result is directed through the objective 6 onto the sample 12 to be cut. The objective 6 is illustrated symbolically in FIG. 2 by a lens. The sample 12, fitted to an object slide 10, is illuminated via the condenser lens 7. The objective 6 produces an imaging beam path 6a which has a greater width than the laser beam 4b after the diaphragm 18.

FIG. 3 illustrates the advantage of a dimmed laser beam 4b which is narrower than the imaging beam path 6a and than a non-dimmed laser beam which fills the entire objective opening 32 by means of which the largest possible beam cross section is defined. The sample 12 has a thickness 30 which can be greater than the depth of focus of the objective 6 used. The user is able to focus on different planes in the sample 12 in order to find points relevant for the further examination.

If the sample 12 is cut with a non-dimmed laser beam whose cross section corresponds to the objective opening 32 of the objective 6, a maximum laser aperture is produced by the objective 6 and is equal to the objective aperture 34. By means of the maximum laser aperture produced, a maximum cutting channel 34b with a width 34a is produced in the sample 12.

If, however, the sample 12 is cut according to the invention with the dimmed laser beam 4b, then the objective 6 produces a reduced laser aperture 36, which produces a reduced cutting channel 36b with a width 36a in the sample 12. The smaller the diameter of the laser beam used for cutting, the more accurately can the cutting operation be carried out.

The fact that the diaphragm 18 for limiting the laser beam cross section before the optical system 16 is arranged outside the observation beam path ensures that the depth of focus of the objective 6 for observing the sample 12 remains unchanged during the cutting operation, irrespective of the set laser aperture. As a result, the image quality is also maintained during the cutting operation.

In order to optimize the cutting quality still further, it is necessary for the diaphragm 18 limiting the laser beam 4a to be matched to the thickness 30 of the sample 12 to be cut. A first possibility is for the diaphragm 18 required for an optimal cut to be determined from a table (not illustrated), and for the diaphragm to be set manually by the user.

In a further exemplary embodiment, the diaphragm 18 required for an optimum cut can be determined by the computer 22 from a stored table (not illustrated). The setting of the diaphragm 18 is then carried out automatically by the computer 22. For this propose, appropriate signals are sent by the computer 22 to the motor 20, which brings about the adjustment of the diaphragm 18.

A further possibility for an optimum cut is for the computer 22 with an image evaluation system (not illustrated) to be attached to the microscope 1 in such a way that individual system parameters, such as the laser intensity, the focal position of the laser beam and the size of the diaphragm 18 are automatically set to an optimum. The setting can be changed automatically, even during the cutting operation, in order to take account of possible thickness fluctuations in the sample 12.

The invention has been described by considering an exemplary embodiment. However, those skilled in the art can perform changes and modifications without departing from the area of protection of the following claims.

### List of Designations

1 Microscope

2 Working table

3 Illumination system

4 Laser

4a Laser beam

4b Dimmed laser beam

5 Microscope stand

6 Objective

6a Imaging beam path

7 Condenser lens

8 Eyepiece

10 Object slide

12 Sample

14 Optical axis

16 Optical system

18 Diaphragm

20 Motor

22 Computer

24 Camera

26 Monitor

30 Thickness of the sample

32 Objective opening

34 Objective aperture

34a Width of the maximum cutting channel

34b Maximum cutting channel

36 Leaser aperture

36a Width of the reduced cutting channel

36b Reduced cutting channel

1. A method for laser cutting microscopic samples, characterized by the following steps:
a) introducing an object slide (10) with a sample (12) to be cut into a microscope (1) which comprises at least one objective (6);
b) with the objective (6), determining an area of the sample (12) to be cut out;
c) defining a cutting line around the area;
d) producing a dimmed laser beam (4b) by means of a diaphragm (18), so that the diameter of said beam is reduced in such a way that a laser aperture (36) produced by the objective (6) is smaller than the objective aperture (34) of the objective (6) itself; and
e) cutting the sample (12) along the defined cutting line.

2. The method as claimed in claim 1, characterized in that the definition of the cutting line is carried out on an image of the sample (12) displayed on a monitor (26), by a mouse pointer being used to move around the area of the sample (12) to be cut out.

3. The method as claimed in claim 1, characterized in that a camera (24) is provided, via which the cutting operation of the laser (4) is controlled and monitored.

4. The method as claimed in claim 3, characterized in that the diaphragm (18) required for an optimum cut is determined from a table, and in that the diaphragm (18) is set manually by the user.

5. The method as claimed in claim 3, characterized in that a computer (22) with an image evaluation system is connected to the microscope (1) in such a way that individual system parameters, such as the laser intensity, the focal position of the laser beam and the size of the diaphragm (18), for example, are automatically set to an optimum.

6. The method as claimed in claim 5, characterized in that the diaphragm (18) required for an optimum cut is determined by the computer (22) from a stored table, and in that the setting of the diaphragm is carried out automatically by the computer (22) via a motor (20).

7. A device for laser cutting microscopic samples comprises:
a) a microscope (1) with at least one objective (6) for observing a sample (12) to be cut, the objective (6) defining an optical axis (14) and an objective aperture (34),
b) a laser (4), which produces a laser beam (4a), and
c) at least one optical system (16), which injects the laser beam (4a) into the objective (6), characterized in that a diaphragm (18) is provided, which produces a dimmed laser beam (4b), a laser aperture (36) produced by the objective (6) being smaller than the objective aperture (34) of the objective (6).

8. The device as claimed in claim 7, characterized in that the size of the diameter of the laser beam (4a) can be varied via a variable diaphragm (18).

9. The device as claimed in claim 7, characterized in that an illumination system (3) is provided, which illuminates the sample (12).

10. The device as claimed in claim 9, characterized in that the illumination system (3) transilluminates the sample (12).

11. The device as claimed in claim 7, characterized in that the optical system (16) comprises at least one dichromatic splitter.

12. The device as claimed in claim 7, characterized in that a camera (24) is provided, via which the cutting operation of the laser (4) can be controlled and monitored.

13. The device as claimed in claim 12, characterized in that the diaphragm (18) required for an optimum cut can be determined from a table, and in that the diaphragm (18) can be set manually by the user.

14. The device as claimed in claim 12, characterized in that a computer (22) with an image evaluation system is connected to the microscope (1) in such a way that individual system parameters, such as the laser intensity, the focal position of the laser beam and the size of the diaphragm (18), for example, can be adjusted.

15. The device as claimed in claim 14, characterized in that the diaphragm (18) required for an optimum cut can be determined by the computer (22) from a stored table, and in that the setting of the diaphragm (18) is carried out automatically by the computer (22).
